(19) United States
(12) Patent Application Publication
(10) Pub. No.: US 2018/0147177 A1
(43) Pub. Date: May 31, 2018

(54) Antibacterial Compositions Comprising Copper Oxo-Hydroxide Nanoparticles and Their Uses as Biocidal Agents

(71) Applicant: Medical Research Council, Swindon Wiltshire (GB)

(72) Inventors: Carlos André Passos BASTOS, Cambridge (GB); Sylvaine Francoise Aline BRUGGRABER, Cambridge (GB); Nuno Jorge Rodrigues FARIA, Milton Ernest (GB); Jonathan Joseph POWELL, Cambridge (GB)

(73) Assignee: Medical Research Council, Swindon Wiltshire (GB)

(21) Appl. No.: 15/567,419

(22) PCT Filed: Apr. 22, 2016
(86) PCT No.: PCT/EP2016/059074

§ 371 Date: Oct. 18, 2017

(30) Foreign Application Priority Data
Apr. 24, 2015 (GB) 1507002.2

(51) Int. Cl. A61K 31/30; A61K 9/00; A61P 31/04

(52) U.S. Cl. CPC A61K 31/30; A61K 9/0017; A61P 31/04; A61K 9/0024

(57) ABSTRACT

Antibacterial compositions comprising nanoparticles formed from copper oxo-hydroxide are described that are capable of delivering biocidal concentrations of copper, typically in the form of free copper ions (Cu²⁺). The nanoparticle compositions generally comprise small particles, typically having mean diameters in the range of 1-100 nm, having comparatively high surface area-to-volume ratio and enhanced reactivity compared to the corresponding bulk counterpart materials and which are sufficiently labile to release the free copper efficiently.

### FIELD OF THE INVENTION

The present invention relates to antibacterial compositions comprising copper oxo-hydroxide nanoparticles, and in particular to ligand-modified copper oxo-hydroxide nanoparticles and their uses as antibacterial agents capable of delivering soluble biocidal copper. The present invention further relates to medical uses of the ligand-modified copper oxo-hydroxide nanoparticles, in particular for wound healing or the treatment or prevention of microbial infection.

### BACKGROUND OF THE INVENTION

Development of new antimicrobials has progressively slowed down since the 1980s, leaving a bleak scenario in the face of emerging multi-drug resistant pathogens. So called ‘superbugs’ are increasingly recognised as a global threat to public health, driving exploration of new antimicrobials—including inorganic agents, such as those based on copper and silver. These metals have had historical usage and, significantly, are hypothesised to act via a multiplicity of biocidal mechanisms—which could potentially enhance clinical longevity by requiring microorganisms to undergo multiple mutations to gain resistance. Of the two metals, silver has shown greater antimicrobial efficacy: however, cost, in vivo toxicity and chemical instability are likely to limit its utility for clinical applications such as the healing of infected wounds. Copper, whilst less efficacious, is inexpensive and, being an essential micronutrient, is better tolerated by man, allowing greater doses to be used. However, owing to its lower biocidal efficacy, the development of delivery systems which maximise bioavailability of the free copper is critical to its use in clinical settings.

GB 1600449 (Mooney Chemicals Inc.) relates to resin or soap-like substances in which crystalline metal oxide particles are surrounded in an amorphous matrix of organic molecules in a stoichiometric manner to produce metal oxide compositions that can be dissolved in non-polar (oil-like) solvents, mainly for use in catalysis. GB 1600449 demonstrates that these compositions retain unmodified crystallite cores by X ray diffraction that shows that the organic molecules are coated on the surface of the particles rather than being incorporated inside them.

WO 2008/096130 (Medical Research Council) describes ligand-modified poly oxo-hydroxy metal ion materials and their uses are disclosed, in particular for nutritional, medical, cosmetic or biologically related applications such as the treatment of a deficiency related to a component of the material such as anaemia or for the removal of an endogenous substance capable of binding to the material. Examples of these types of materials for use as phosphate binding materials are described in WO 2010/015827.

WO 2012/101407 relates to oxygen sensors for use in product packaging for storing an article in a packaging envelope under modified atmosphere conditions, in which the oxygen sensors are based on metal oxo-hydroxides that are optionally modified with one or more ligands. The sensors may be present in a hydrated, oxygen permeable matrix, for example formed from a material, such as gelatine.

DE 20205014332 relates to organometallic nanopowders containing chemically reactive groups and their use in the formation of polymeric composites.

The development of approaches for the effective delivery of antimicrobial metal ions such as copper, remains an unresolved problem in the art, especially for use in a clinical setting.

### SUMMARY OF THE INVENTION

Broadly, the present invention relates to nanoparticles formed from copper oxo-hydroxide that are capable of delivering biocidal concentrations of copper, typically in the form of free copper ions (Cu²⁺). The nanoparticle compositions of the present invention achieve this result by providing small particles, typically having mean diameters in the range of 1 nm to 100 nm, and more preferably in the range of 1 nm to 10 nm, having comparatively high surface area-to-volume ratio and enhanced reactivity compared to the corresponding bulk counterpart materials and which are sufficiently labile to release the free copper efficiently, enabling them to act as pharmaceutical or antibacterial compositions, unlike prior art copper nanoparticles. In preferred embodiments, this is achieved by through ligand modification of the copper oxo-hydroxide in which one or more ligands are non-stoichiometrically substituted for the oxo or hydroxy groups of the copper oxo-hydroxide. The experiments described herein demonstrate that the copper oxo-hydroxide nanoparticles are as effective as antibacterial agents and are superior compared to commercially available copper oxide (CuO) nanoparticles, silicate stabilised copper hydroxide nanoparticles and copper complexes with strong chelating agents, such as EDTA. The copper oxo-hydroxide nanoparticle compositions of the present invention are preferably modified with carboxylic acid ligands, or ionised forms thereof, such as tartarate and adipate.

Accordingly, in a first aspect, the present invention provides a antibacterial composition comprising ligand-modified copper oxo-hydroxide nanoparticles, wherein the copper oxo-hydroxide nanoparticles have a structure in which the one or more ligands are non-stoichiometrically substituted for the oxo or hydroxy groups, wherein the one or more ligands comprise a carboxylic acid ligand, or an ionised form thereof. As the copper oxo-hydroxide nanoparticles have a polymeric structure in which the ligands are distributed within the solid phase structure of the copper oxo-hydroxide, rather than simply being coated or physically adsorbed on the surface of the particles of copper oxo-hydroxide, the present inventors believe that the inclusion of the ligands helps to modulate the dissolution of the nanoparticles to provide free soluble copper ions available for biocidal use. It is preferred that the copper oxo-hydroxide nanoparticles have one or more reproducible physico-chemical properties, for example dissolution profile, percentage of soluble copper made available as a function of total copper present in the nanoparticles and/or biocidal activity of the nanoparticles in a bacterial growth inhibition assay and/or retention of lability upon resuspending a composition that has been dried.

In a further aspect, the present invention provides a copper oxo-hydroxide nanoparticle composition as described herein for use as an antibacterial agent.

In a further aspect, the present invention provides a copper oxo-hydroxide nanoparticle composition as described herein for use in a method for the treatment or prevention of a microbial infection, and more preferably wherein the microbial infection is a bacterial infection. In aspects of the present invention relating to the medical uses of the copper oxo-hydroxide nanoparticle compositions, the composition may be employed for treating a human or animal subject.

In a further aspect, the present invention provides a pharmaceutical composition comprising ligand-modified copper oxo-hydroxide nanoparticles as described herein, and a pharmaceutically acceptable carrier.

In a further aspect, the present invention provides the use of copper oxo-hydroxide nanoparticle composition as described herein for the preparation of a medicament for the treatment or prevention of bacterial infection or the treatment of wounds.

In a further aspect, the present invention provides a method of treating or preventing a bacterial infection, the method comprising administering to a patient in need of treatment a therapeutically effective amount of copper oxo-hydroxide nanoparticle composition as described herein.

In a further aspect, the present invention provides an article that has been coated or treated with an antibacterial composition of the present invention.

In a further aspect, the present invention provides a process for producing a copper oxo-hydroxide nanoparticle composition according to the present invention, the process comprising:
(a) mixing the solution comprising Cu²⁺ and a carboxylic acid ligand, and optionally one or more further ligands or reaction components, in a reaction medium at a first pH(A) at which the components are soluble;
(b) changing the pH(A) to a second pH(B) to cause a solid precipitate or a colloid of the copper oxo-hydroxide nanoparticle composition to be formed;
(c) separating, and optionally drying and/or formulating, the copper oxo-hydroxide nanoparticle composition produced in step (b).

In a further aspect, the present invention provides a composition comprising ligand-modified copper oxo-hydroxide nanoparticles, wherein the copper oxo-hydroxide nanoparticles have a structure in which the one or more ligands are non-stoichiometrically substituted for the oxo or hydroxy groups, wherein the one or more ligands comprise a carboxylic acid ligand, or an ionised form thereof, as obtainable by the above process.

In a further aspect, the present invention provides an article having a surface treated to include ligand-modified copper oxo-hydroxide nanoparticles of the present invention, wherein the nanoparticles provide the surface of the article with antibacterial properties. Examples of articles treatable in this way, such as medical equipment, bandages and dressings, are provided below.

It will be understood that the coated substrates of the invention may be for use in a method of medical treatment, for example for the treatment and/or prophylaxis of microbial infection or the treatment of wounds. The substrate may also be useful for the treatment and/or prophylaxis of skin disorders or disorders of mucous membranes. In a further aspect, then, the present invention provides use of a ligand modified copper oxo-hydroxide nanoparticle compositions in the manufacture of a medicament for the treatment and/or prophylaxis of microbial or bacterial infection. The present invention also provides use of a ligand modified copper oxo-hydroxide nanoparticle composition in the manufacture of a medicament for the treatment and/or prophylaxis of skin disorders or disorders of mucous membranes. It is understood that the medicament may be a coating or coated substrate of the present invention, for example a coated wound dressing, or a coated medical device such as an implantable medical device, for example a stent.

Embodiments of the present invention will now be described by way of example and not limitation with reference to the accompanying figures and examples. FIGS. 1-6 are provided for comparative purposes.

### BRIEF DESCRIPTION OF THE FIGURES

FIG. 1. Growth curves of E. coli with exposure to copper chloride at 0 to 50 ppm Cu. Bacterial growth was followed through the measurement of optical density (OD) over time (top). Copper chloride solubility in the bacterial growth medium over the period of the assay (bottom). Error bars represent standard deviations (n=3).

FIG. 2. (A) CuO and CuSi NPs (as per experimental examples) in water at ca. 1270 ppm, immediately after being dispersed (0 h) and one hour after standing without agitation, showing the formation of large CuO agglomerates (black sediment), unlike CuSiNPs that remained stable in suspension. (B) Zeta potential of CuO NPs in a water suspension at ca. 1270 ppm Cu. Particle size distribution (C) and zeta potential (D) of CuSiNPs, both analysed at pH 12 in water at ca. 1200 ppm copper. Error bars represent standard deviations (n=3).

FIG. 3. E. coli growth curves after exposure to CuO NPs (A) and CuSi NPs at 50 ppm Cu (B), and their respective bacterial growth inhibition in comparison to CuCl₂, soluble copper control (C). Dissolution of CuSi NPS and CuO NPs in bacterial culture medium, at 50 ppm Cu (D). Error bars represent standard deviations (n=3).

FIG. 4. Comparison of E. coli growth inhibition with levels of nanoparticulate and soluble copper in the bacterial culture medium, at 3 different concentrations of copper (12.5, 25 and 50 ppm Cu) after 4 hours of incubation with CuO NPs (Top) and CuSi NPs (bottom).

FIG. 5. Dispersible Cu in a MOPS buffer at pH 7.4±0.2 upon dilution of CuCl₂ to a range of concentrations from 10 to 500 ppm (n=3).

FIG. 6. (A) Solubility of Cu-EDTA stocks at pH 7.5±0.2 in water at ca. 1270 ppm Cu. Error bars represent standard deviations of two analytical replicates. (B) E. coli growth inhibition after incubation with CuEDTA complexes at different ratios. Note that negative values represent an increase in initial growth compared to copper-free cultures. (C) Dispersible copper in a MOPS buffer at pH 7.4±0.2 upon dilution of CuSi nanoparticles to a range of concentrations from 10 to 500 ppm. Error bars represent standard deviations (n=3).

FIG. 7. Characterisation of CuTartAd nanoparticles (prepared as per experimental examples). A—TEM analysis of a suspension, as prepared, at pH 8 containing ca. 2500 ppm Cu (suspension was dropcast on a TEM grid). B—Hydrodynamic particle size distribution of the same nanoparticles analysed by Dynamic Light Scattering showing a mean size of 3.72±0.04 nm. C. Zeta Potential of CuTartAd NPs at pH 8, ca. 1000 ppm Cu. D. XRD spectrum of amorphous CuTartAd nanoparticles. Peaks in red correspond to halite (crystalline NaCl), which was formed by neutralisation of an acidic chloride-containing solution with sodium hydroxide. Error bars represent standard deviations of three analytical replicates.

FIG. 8. A) Dissolution profile of CuTartAd NPs in bacterial culture medium at 12.5, 25 and 50 ppm Cu; Error bars represent standard deviations (n=3). B) Dispersible copper in MOPS buffer at pH 7.4±0.2 upon dilution of CuTartAd NPs to a range of concentrations from 10 to 500 ppm. Error bars represent standard deviations (n=3).

FIG. 9. E. coli (top) and S. aureus (bottom) growth inhibition after incubation with soluble Cu and CuTartAd nanoparticles both at 50 ppm Cu. Error bars represent standard deviations (n=2).

FIG. 10. HEC matrix containing CuTartAd NPs at 250 ppm Cu (A). Release of Cu from HEC matrices containing 250 ppm Cu. Error bars represent standard deviations (n=3). This assay consisted of exposing the copper-containing HEC—with specific surface area (7.1 cm²)—to a bicarbonate buffered solution at pH 7.0±0.2, following copper concentration over time.

FIG. 11. XRD spectrum of the unmodified copper hydroxide synthesized for comparative purposes (Example 4.N6) was also obtained (bottom). The latter showed a crystalline pattern corresponding to paratacamite, a copper hydroxide of chemical formula Cu₂(OH)₃Cl in which a chlorine atom was incorporated in the mineral structure (bottom).

FIG. 12. Cell proliferation of skin fibroblasts (cell line CCD-25Sk) upon exposure to CuCl₂, AgNO₃ and tartrate adipate modified copper oxo-hydroxide nanoparticles (CuTartAd NPs) for 48 hours.

### DETAILED DESCRIPTION

Copper Oxo-Hydroxide Nanoparticles Compositions

The production and characterisation of solid ligand-modified poly oxo-hydroxy metal ion materials, and in particular materials based on ferric iron oxo-hydroxide, are described in our earlier applications WO 2008/096130 and WO 2010/015827, both of which are incorporated by reference. Corresponding processes were adapted in the work reported in the present application to provide the ligand modified copper oxo-hydroxide nanoparticle compositions of the present invention that have uses in antibacterial and antimicrobial applications, for example for promoting wound healing.

In general, this class of materials may be represented by the non-stoichiometric formula (MₓL_{y}(OH)ₙ), where M represents one or more metal ions, L represents one or more ligands and OH represents oxo or hydroxy groups, depending on whether the groups are bridging (oxo groups) or surface groups in the solid oxo-hydroxide material. As is well known in the art, non-stoichiometric compounds are chemical compounds with an elemental composition that cannot be represented by a ratio of well-defined natural numbers, i.e. the x, y and n subscripts in the formula above will not necessarily all be natural numbers, even though the materials can be made in a reproducible manner and have consistently reproducible properties. Preferably, the ligand modified copper oxo-hydroxides of the present invention have a polymeric structure in which the ligands are substantially randomly substituted for the oxo or hydroxy groups. This provides copper oxo-hydroxide nanoparticles having one or more reproducible physicochemical properties, for example compositions having one or more of a mean particle size diameter in the range of about 1 nm to about 100 nm (for example as determined by dynamic light scattering, see section 1.2.1), a reproducible dissolution profile, compositions in which the nanoparticles are substantially amorphous (for example as determined using X-ray diffraction or transmission electron microscopy, see sections 1.2.3 and 1.2.4) and/or compositions in which the nanoparticles have demonstrable metal-ligand bonding (for example as determined using infra-red spectroscopy). Additionally or alternatively, the copper oxo-hydroxide nanoparticle compositions are capable of releasing a percentage of soluble copper that is preferably at least 25% of the total copper present in the composition, more preferably at least 30%, more preferably at least 40% and most preferably at least 50%. The release of soluble copper may be measured in a free copper release assay (e.g. as described in the examples below). The biocidal properties of the copper oxo-hydroxide nanoparticle compositions may be measured using a bacterial growth inhibition assay and preferably achieves at least 50% bacterial growth inhibition, more preferably at least 60% bacterial growth inhibition, more preferably at least 70% bacterial growth inhibition, and more preferably at least 90% bacterial growth inhibition under standardised conditions. In a preferred embodiment, full (100%) inhibition of E. coli growth is achieved using the antimicrobial compositions of the present invention, for example in an assay in which E. coli was exposed to the ligand modified copper oxo-hydroxide nanoparticles for 6 hours with copper concentrations above 25 mg/L fully inhibiting (100%) E. coli growth in these specific conditions. A further example of a suitable growth inhibition assay is provided in section 1.3.2.

Typically, the metal ion (e.g. Cu²⁺) will originally be present in the form of a salt that in the preparation of the materials may be dissolved and then induced to form poly oxo-hydroxy co-complexes with ligand (L). As described below, other metal ions may be present in addition to copper ions (Cu²⁺). While not wishing to be bound by any particular theory, the present inventors believe that in these materials, and in the ligand modified copper oxo-hydroxide nanoparticles of the present invention, some of the ligand used to modify the metal oxo-hydroxide is integrated within the solid phase through formal M-L bonding, i.e. not all of the ligand (L) is simply trapped or adsorbed in the bulk material and/or is adsorbed or coated on the surface of the particles of the metal oxo-hydroxide material. The bonding of the metal ion in the materials can be determined using physical analytical techniques such as infrared spectroscopy where the spectra will have peaks characteristic of the bonds between the metal ion and the ligand (L), as well as peaks characteristic of other bonds present in the material such as M-O, O—H and bonds in the ligand species (L). Alternatively or additionally, the ligand species may be introduced into the solid phase structure by the substitution of oxo or hydroxyl groups by ligand molecules in a manner that decreases overall order in the solid phase material, so that the materials have a more amorphous nature compared, for example, to the structure of the corresponding unmodified copper hydroxide. The presence of a more disordered or amorphous structure can readily be determined by the skilled person using techniques well known in the art. One exemplary technique is Transmission Electron Microscopy (TEM). High resolution transmission electron microscopy allows the crystalline pattern of the material to be visually assessed. It can indicate the primary particle size and structure (such as d-spacing), give some information on the distribution between amorphous and crystalline material. This may be especially apparent using high angle annular dark field aberration-corrected scanning transmission electron microscopy due to the high contrast achieved while maintaining the resolution, thus allowing the surface as well as the bulk of the primary particles of the material to be visualised.

The copper oxo-hydroxide nanoparticles disclosed herein use copper ions (Cu²⁺) to provide compositions that are capable of delivering biologically effective concentrations of biocidal copper, for example for use as an antibacterial or antimicrobial agents. The compositions of the present invention may further have the advantage of being biologically compatible and non toxic in view of the general physiological tolerance to copper.

By way of background, it is well known in the art that copper oxides, hydroxides and oxo-hydroxides are composed of Cu²⁺ together with O and/or OH and are collectively referred to in this patent and known in the art as “copper oxo-hydroxides”. In addition to the presence of copper ions (Cu²⁺), other metal ions may be present such as metal cations selected from Ca²⁺, Mg²⁺, Ag⁺, Al³⁺, Fe³⁺ and/or Zn²⁺. In particular, it may be desirable to include further metal cations with antimicrobial properties, such as Ag⁺. A further preferred type of materials include Zn²⁺, in addition to copper ions.

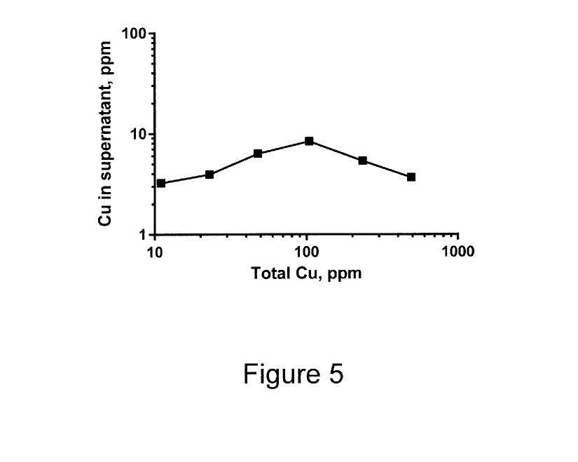

The copper oxo-hydroxide nanoparticles of the present invention are based on the development of compositions designed for optimal delivery of soluble copper, for example for use in applications where antibacterial activity of soluble copper is desirable. The comparative examples herein show that when dispersed at the concentrations that are required in clinical formulations, common copper salts tend to be precipitated as large, and biocidally inactive, copper hydroxides (as shown in FIG. 5). Moreover, while the addition of complexing agents (e.g. EDTA) prevents the formation of such agglomerates, and is capable of keeping copper in solution, these preparations showed modest inhibition of bacterial growth due to the limited bioavailability of complexed copper ions. These experiments showed that despite copper ions being the active form responsible to biocidal activity that copper salts are not a good way of delivering them as the salt forms tend to convert to insoluble copper hydroxides. The present inventors realised that both of these approaches are undesirable as the copper ions are biologically unavailable, either by being present in agglomerates or by being strongly complexed by agents, such as EDTA.

Accordingly, the present invention concerns nanoparticulate systems for the delivery of free copper ions by functionalising copper oxo-hydroxide nanoparticles with ligands, for example dietary ligands such as carboxylic acids or amino acids. In a preferred approach, the mineral phase of copper oxo-hydroxide nanoparticles was modified through the use of carboxylate ligands, such as tartrate, gluconate, adipate and/or glutathione, which conferred negative surface charge, and stabilised the nanoparticles in aqueous environments.

Preferably, the copper oxo-hydroxide nanoparticles of the present invention have mean diameter ranges 1 to 100 nm, 1 to 50 nm, 1 to 20 nm, 1 to 10 nm. The size of the particles of copper oxo-hydroxide nanoparticles can be determined using techniques well known in the art such as dynamic light scattering, as demonstrated in the examples in section 1.2.1. By way of example, this may be carried out using a Zetasizer NanoZS (Malvern Instruments). In a typical experiment, 0.5 to 1 ml of a suspension of copper oxo-hydroxide nanoparticles may be transferred into a small disposable cuvette at room temperature (20±2° C.) and measurements were carried out using the following settings: material refractive index 0.192, absorption 0.1, dispersant refractive index 1.330, viscosity 1.00331 mPa·s.

Without modification, the primary particles of the materials used herein have metal oxide cores and metal hydroxide surfaces and within different disciplines may be referred to as metal oxides or metal hydroxides. The use of the term ‘oxo-hydroxy’ or ‘oxo-hydroxide’ is intended to recognise these facts without any reference to proportions of oxo or hydroxy groups. Hydroxy-oxide could equally be used therefore. For the avoidance of doubt, copper hydroxide also includes various chloride-doped polymorphs; in particular, Cu₂(OH)₃Cl is a copper hydroxide derivative in which a chlorine atom was incorporated in the crystalline structure that presents four types of mineral phase: atacamite, botallackite, paratacamite and clinoatacamite. The present inventors believe that the copper oxo-hydroxide nanoparticles compositions of the present invention are altered at the level of the primary particle of the metal oxo-hydroxide with at least some of the ligand L being introduced into the structure of the primary particle, i.e. leading to doping of the primary particle by the ligand molecules. This may be contrasted with the formation of nano-mixtures of metal oxo-hydroxides and an organic molecule in which the structure of the primary particles is not so altered and the organic ligand is only coated or adsorbed on the surface of the particles, as happens when the metal oxo-hydroxide particles are preformed prior to being contacted with the ligand.

The primary particles of the ligand-modified poly oxo-hydroxy metal ion materials described herein may conveniently be produced by precipitation. The use of the term precipitation often refers to the formation of aggregates of materials that do separate from solution by sedimentation or centrifugation. Here, the term “precipitation” is intended to describe the formation of all solid phase material, including aggregates as described above and solid materials that do not aggregate but remain as non-soluble moieties in suspension, whether or not they be particulate or nanoparticulate (colloidal or sub-colloidal). These latter solid materials may also be referred to as aquated particulate solids.

In the present invention, reference may be made to the modified metal oxo-hydroxides having polymeric structures that are not generally crystalline and so have three dimensional polymeric or cross-linked structures that generally form above the critical precipitation pH. As used herein, this should not be taken as indicating that the structures of the materials are polymeric in the strict sense of having a regular repeating monomer unit because, as has been stated, ligand incorporation is, except by co-incidence, non-stoichiometric. The ligand species is introduced into the solid phase structure by substituting for oxo or hydroxy groups leading to a change in solid phase order. In some cases, for example the production of the copper oxo-hydroxide nanoparticle compositions exemplified herein, the ligand species L may be introduced into the solid phase structure by the substitution of oxo or hydroxy groups by ligand molecules in a manner that decreases overall order in the solid phase material. While this still produces solid ligand modified poly oxo-hydroxy metal ion materials that in the gross form have one or more reproducible physico-chemical properties, the materials have a more amorphous nature compared, for example, to the structure of the corresponding unmodified metal oxo-hydroxide. The presence of a more disordered or amorphous structure can readily be determined by the skilled person using techniques well known in the art. One exemplary technique is Transmission Electron Microscopy (TEM). High resolution Transmission Electron Microscopy allows the crystalline pattern of the material to be visually assessed. It can indicate the primary particle size and structure (such as d-spacing), give some information on the distribution between amorphous and crystalline material. Using this technique, it is apparent that the chemistry described above increases the amorphous phase of our described materials compared to corresponding materials without the incorporated ligand. This may be especially apparent using high angle annular dark field aberration-corrected scanning transmission electron microscopy due to the high contrast achieved while maintaining the resolution, thus allowing the surface as well as the bulk of the primary particles of the material to be visualised.

The combination of these physicochemical properties described above promotes rapid release of copper ions, and as shown in the examples translates into high bactericidal efficacy against a broad range of both gram negative and gram positive bacteria. Importantly, oxo-hydroxides modified with carboxylates, unlike copper salts (such as CuCl₂) or commercial copper nanoparticles, were able to release copper at biocidal levels when incorporated in a delivery matrix such as hydroxyethyl cellulose gel (an example of a topical delivery matrix), showing that ligand functionalisation can be used for the development of topical biocides or to provide a composition that is capable of providing an antibacterial coating for articles.

Examples of properties that can be usefully modulated using the present invention include: dissolution (rate, pH dependence and [Cu] dependence), disaggregation, adsorption and absorption characteristics, reactivity-inertness, melting point, temperature resistance, particle size, magnetism, electrical properties, density, light absorbing/reflecting properties, hardness-softness, colour and encapsulation properties. In this context, a property or characteristic may be reproducible if replicate experiments are reproducible within a standard deviation of preferably ±10%, and more preferably ±5%, and even more preferably within a limit of ±2%. In particular, the present inventors have found that properties of the copper oxo-hydroxide nanoparticles such as lability are retained upon resuspending compositions that have been dried, for example for storage.

The dissolution profile of the ligand modified copper oxo-hydroxide nanoparticles compositions can be represented by different stages of the process, namely disaggregation and dissolution. The term dissolution is used to describe the passage of a substance from solid to soluble phase. More specifically, disaggregation is intended to describe the passage of the materials from a solid aggregated phase to an aquated phase that is the sum of the soluble phase and the aquated particulate phase (i.e. solution plus suspension phases). Therefore, the term dissolution as opposed to disaggregation more specifically represents the passage from any solid phase (aggregated or aquated) to the soluble phase.

The Ligand (L)

In the ligand modified copper oxo-hydroxide nanoparticles compositions represented by the formula (MₓL_{y}(OH)ₙ), L represents one or more ligands or anions, such as initially in its protonated or alkali metal form, that can be incorporated into the solid phase ligand-modified poly oxo-hydroxy metal ion material. In the materials described herein, at least one of the ligands is a carboxylic acid ligand, or an ionised form thereof (i.e., a carboxylate ligand), such as tartarate (or tartaric acid), gluconate (or gluconic acid), adipate (or adipic acid), glutathione and/or an amino acid and/or a sugar acid.

Preferably, the ligand is a mono or dicarboxylic acid ligand, and may be represented by the formula HOCH₂—R₁—COOH or HOOC—R₁—COOH (or an ionised form thereof), where R₁ is an optionally substituted C₁₋₁₀ alkyl, C₁₋₁₀ alkenyl or C₁₋₁₀ alkynyl group. In general, the use of ligands in which R₁ is a C₁₋₁₀ alkyl group, and more preferably is a C₂₋₆ alkyl group, is preferred. Preferred optional substituents of the R₁ group include one or more hydroxyl groups, for example as present in malic acid. In preferred embodiments, the R₁ group is a straight chain alkyl group. A more preferred group of carboxylic acid ligands include tartaric acid (or tartarate), gluconate (or gluconic acid), adipic acid (or adipate), glutaric acid (or glutarate), pimelic acid (or pimelate), succinic acid (or succinate), and malic acid (or malate), and combinations thereof. Whether the carboxylic acid ligand is present as the acid or is partially or completely ionised and present in the form of a carboxylate anion will depend on a range of factors such as the pH at which the material is produced and/or recovered, the use of post-production treatment or formulation steps and how the ligand becomes incorporated into the poly oxo-hydroxy metal ion material. In some embodiments, at least a proportion of the ligand will be present in the carboxylate form as the material are typically recovered at pH>4 and because the interaction between the ligand and the positively charged iron would be greatly enhanced by the presence of the negatively charged carboxylate ion. For the avoidance of doubt, the use of carboxylic acid ligands in accordance with the present invention covers all of these possibilities, i.e. the ligand present as a carboxylic acid, in a non-ionised form, in a partially ionised form (e.g., if the ligand is a dicarboxylic acid) or completely ionised as a carboxylate ion, and mixtures thereof.

Typically, ligands are incorporated in the solid phase poly oxo-hydroxy metal ion materials to aid in the modification of a physico-chemical property of the solid material, e.g. as compared to a poly oxo-hydroxylated metal ion species in which the ligand(s) are absent. In some embodiments of the present invention, the ligand(s) L may also have some buffering capacity. Examples of ligands that may be employed in the present invention include, but are by no means limited to: carboxylic acids such as tartaric acid, gluconic acid, adipic acid, glutaric acid, malic acid, succinic acid, aspartic acid, pimelic acid, citric acid, lactic acid or benzoic acid; food additives such as maltol, ethyl maltol or vanillin; amino acids such as tryptophan, glutamine, proline, valine, or histidine; and nutrient-based ligands such as folate, ascorbate, pyridoxine or niacin or nicotinamide; sugar acids such as gluconic acid. Typically, two ligands of differing affinities for the metal ion are used in the production of these materials although one, two, three, four or more ligands may be useful in certain applications.

For many applications, ligands need to be biologically compatible under the conditions used and generally have one or more atoms with a lone pair of electrons at the point of reaction. The ligands include anions, weak ligands and strong ligands. Ligands may have some intrinsic buffering capacity during the reaction. Without wishing to be bound by a particular explanation, the inventors believe that the ligands have two modes of interaction: (a) substitution of oxo or hydroxy groups and, therefore, incorporation with a largely covalent character within the material and (b) non-specific adsorption (ion pair formation). These two modes likely relate to differing metal-ligand affinities (i.e. strong ligands for the former and weak ligands/anions for the latter). There is some evidence in our current work that the two types of ligand are synergistic in modulating dissolution characteristics of the materials and, perhaps, therefore, in determining other characteristics of the material. In this case, two ligand types are used and at least one (type (a)) is demonstrable as showing metal binding within the material. Ligand efficacy, probably especially for type (b) ligands, may be affected by other components of the system, particularly electrolyte.

The ratio of the metal ion(s) to the ligand(s) (L) is also a parameter of the solid phase ligand-modified poly oxo-hydroxy metal iron material that can be varied according to the methods disclosed herein to vary the properties of the materials. Generally, the useful ratios of Cu:L will be between 10:1, 5:1, 4:1, 3:1, 2:1 and 1:1 and 1:2, 1:3, 1:4, 1:5 or 1:10, and preferably ratios of Cu to ligand of 1:1 or lower.

Producing and Processing the Copper Oxo-Hydroxide Nanoparticle Compositions

Generally, the copper oxo-hydroxide nanoparticle compositions of the present invention may be produced by a process comprising:
(a) mixing the solution comprising Cu³⁺ and a carboxylic acid ligand, and optionally any further ligands or other components, in a reaction medium at a first pH(A) at which the components are soluble;
(b) changing the pH(A) to a second pH(B) to cause a solid precipitate or a colloid of the copper oxo-hydroxide nanoparticle composition to be formed;
(c) separating, and optionally drying and/or formulating, the copper oxo-hydroxide nanoparticle composition produced in step (b).

Examples of conditions that may be employed include the following using a first pH(A) which is less than 4.0 and the second pH(B) which is between 5.0 and 12.0, and more preferably between 6.0 and 8.0, and carrying out the reaction at room temperature (20-25° C.). In general, it is preferred that in step (a), the solution contains 20 to 100 mM or 1M Cu²⁺ and 50 to 250 mM of a suitable carboxylic acid ligand, and more preferably about 40 mM Cu²⁺ and about 100 mM of the ligand.

The separation of a candidate material may then be followed by one or more steps in which the material is characterised or tested. By way of example, the testing may be carried out in vitro or in vivo to determine one or more properties of the material as described above, most notably its dissolution profile, release of soluble copper and/or antibacterial properties. Alternatively or additionally, the process may comprise chemically, e.g. through a titration process, or physically, e.g. through a micronizing process, altering the final particle size of the copper oxo-hydroxide nanoparticle composition and/or subjecting the composition to one or more further processing steps on the way to producing a final composition, e.g. for administration to a subject. Examples of further steps include, but are not limited to: washing, centrifugation, filtration, spray-drying, freeze-drying, vacuum-drying, oven-drying, dialysis, milling, granulating, encapsulating, tableting, mixing, compressing, nanosizing and micronizing.

In some embodiments, additional steps may be carried out between the initial production of the material and any subsequent step in which it is formulated as a medicament. These additional post-production modification steps may include the step of washing the material, to remove impurities or replace an incorporated ligand with the further ligand.

Hydroxy and Oxo Groups

The present invention may employ any way of forming hydroxide ions at concentrations that can provide for hydroxy surface groups and oxo bridging in the formation of these poly oxo-hydroxy materials. Examples include but are not limited to, alkali solutions such as sodium hydroxide, potassium hydroxide sodium phosphate and sodium bicarbonate, that would be added to increase [OH] in an ML mixture, or acid solutions such as mineral acids or organic acids, that would be added to decrease [OH] in an ML mixture.

The conditions used to produce the copper oxo-hydroxide nanoparticle compositions of the present invention may be tailored to control the physico-chemical nature of the precipitate, or otherwise assist in its collection, recovery or formulation with one or more excipients. This may involve purposeful inhibition of agglomeration, or the used drying or grinding steps to subsequently affect the material properties. However, these are general variables to any such system for solid extraction from a solution phase. After separation of the precipitated material, it may optionally be dried before use or further formulation. The dried product may, however, retain some water and be in the form of a hydrated solid phase ligand-modified poly oxo-hydroxy metal ion material. It will be apparent to those skilled in the art that at any of the stages described herein for recovery of the solid phase, excipients may be added that mix with the ligand-modified poly oxo-hydroxy metal ion material but do not modify the primary particle and are used with a view to optimising formulation for the intended function of the material. Examples of these could be, but are not limited to, glycolipids, phospholipids (e.g. phosphatidyl choline), sugars and polysaccharides, sugar alcohols (e.g. glycerol), polymers (e.g. polyethyleneglycol (PEG)) and taurocholic acid.

Formulations and Uses

The copper oxo-hydroxide nanoparticle composition of the present invention may be formulated for use as antibacterial agents or antimicrobial agents, for example for the treatment or prevention of bacterial or microbial infections. Accordingly, the compositions of the present invention may comprise, in addition to one or more of the solid phase materials of the invention, a pharmaceutically acceptable excipient, carrier, buffer, stabiliser or other materials well known to those skilled in the art. Such materials should be non-toxic and should not significantly interfere with the efficacy of the solid phase materials for the application in question.

The term “antibacterial” as used herein includes the treatment or prevention of infections caused by gram negative and gram positive microorganisms including Escherichia sp., such as E. coli, Staphylococcus sp., such as S. epidermis, S. aureus and meticillin-resistant staphylococcus aureus (“MRSA”), Bacillus sp., such as B. subtilis, Pseudomonas sp., such as P. aeruginosa, Vibrio sp., such as V. fisheri, Streptococcus sp., such as S. pyrogenes and S. pneumoniae, Klebsiella sp., Micrococcus sp., such as M. luteus, Clostridium sp. such as C. difficile, Acinetobacter sp. such as A. baumannii, Mycobacterium sp., such as M. tuberculosis and Salmonella sp, or fungi including Candida sp., such as C. albicans. The term “antimicrobial” as used herein is understood to apply to substances including those which inhibit microbial attachment to surfaces, kill microbes and/or inhibit microbial reproduction. The term “microbe” is understood to include all microorganisms, including bacteria as set out above, as well as fungi such as yeast, archaea and protists. The terms “microbial” and “antimicrobial” should be interpreted accordingly.

The use of the copper oxo-hydroxide nanoparticle compositions of the present invention will very depending on whether the compositions are intended for the treatment or prevention of infection in a human or animal subject, or to provide a surface of an article that is resistant to bacterial or microbial colonisation. Example of the latter application include providing coatings for medical equipment or dressings.

In embodiments in which the compositions are intended for the administration to subject, for example in the treatment of wounds or skin infections, the precise nature of the carrier or other component may be related to the manner or route of administration of the composition, typically via a topical route. This may include formulation of the nanoparticle compositions in a solid, semi-solid or gel matrix or in a liquid carrier such as water, petroleum, animal or vegetable oils, mineral oil or synthetic oil. Examples of carriers include physiological saline solution, dextrose or other saccharide solution or glycols such as ethylene glycol, propylene glycol or polyethylene glycol may be included.

The materials and compositions used in accordance with the present invention that are to be given to an individual are preferably administered in a “prophylactically effective amount” or a “therapeutically effective amount” (as the case may be, although prophylaxis may be considered therapy), this being sufficient to show benefit to the individual clinical state. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, Lippincott, Williams & Wilkins. A composition may be administered alone or in combination with other treatments, either simultaneously or sequentially, dependent upon the condition to be treated.

In one example, the copper oxo-hydroxide nanoparticle compositions of the present invention are formulated in a matrix, for example a hydroxyalkyl cellulose matrix, such as hydroxyethylcellulose (HEC) or hydroxymethylcellulose (HMC), or a polyalkylene glycol matrix, such as PEG. Some of these matrices are cellulose derivatives that have been widely used in health care products and cosmetics and have the advantage that they do not require further processing (e.g. heating and drying) of nanoparticles during matrix preparation, thereby having a minimal effect on the antibacterial or antimicrobial properties of the nanoparticle compositions.

In other embodiments, the copper oxo-hydroxide nanoparticle compositions of the present invention may be formulated for topical administration, e.g. in the form of a solid or semi-solid ointment useful in the treatment of wounds, ulcers or the treatment or prevention of bacterial infection. In such applications, polyalkylene glycols are well suited for topical delivery of the materials as they form a cream or an ointment and is available in a range of different molecular weights, allowing the tailoring of viscosity and other physical parameters that may be desirable in the final ointment. The application of the present invention to topical products has therapeutic use for wound healing and as in anti-infective compositions.

In all aspects of the present invention in which the compositions are formulated for administration to a subject, it is preferred that the pH of the composition or a formulation containing it is raised to a physiological pH, preferably to a pH between 5.0 and 9.0, and more preferably to a pH of between 6.0 and 8.5. The examples show that the compositions of the present invention are capable of making free copper bioavailable under these conditions. Conveniently, this may be done by adding a base, such as sodium hydroxide or sodium carbonate. The aim of this is so that administration to a subject will not result in unintended clinical outcomes, such as pain or inflammation.

An effective amount of copper oxo-hydroxide nanoparticle compositions herein may be formulated for topical application, e.g. to the skin, teeth, nails or hair. These compositions can be in the form of creams, lotions, gels, suspensions, dispersions, microemulsions, nanodispersions, microspheres, hydro gels, emulsions (oil-in-water and water-in-oil, as well as multiple emulsions) and multilaminar gels and the like (see, for example, The Chemistry and Manufacture of Cosmetics, Schlossman et al., 1998), and may be formulated as aqueous or silicone compositions or may be formulated as emulsions of one or more oil phases in an aqueous continuous phase (or an aqueous phase in an oil phase). The type of carrier utilized in the present invention depends on the properties of the topical composition. The carrier can be solid, semi-solid or liquid. Suitable carriers are liquid or semi-solid, such as creams, lotions, gels, sticks, ointments, pastes, sprays and mousses. Specifically, the carrier is in the form of a cream, an ointment, a lotion or a gel, more specifically one which has a sufficient thickness or yield point to prevent the particles from sedimenting. The carrier can itself be inert or it can possess benefits of its own. The carrier should also be physically and chemically compatible with the antibacterial composition or other ingredients formulated in the carrier. Examples of carriers include water, hydroxyethyl cellulose, propylene glycol, butylene glycol and polyethylene glycol, or a combination thereof.

In addition to the therapeutic use of the ligand modified copper oxo-hydroxide nanoparticle composition, they may also be applied as antimicrobial or antibacterial coatings to articles, for example coatings on substrates which comprise woven fabric, non-woven fabric, plastic, glass and/or metal. The antimicrobial nature of the coatings makes them particularly suitable to be applied to substrates for use in medical or personal care applications. In particular, the coatings are particularly useful on substrates which are in contact with the body, for example with skin or mucous membrane, in normal use, for example dressings, bandages and plasters.

For example, microbial growth is a particular problem when skin or mucous membrane is covered, for example by a wound dressing, nappy or underwear. As soon as skin or mucous membrane becomes covered, the environmental conditions for microbial growth improve. Microbes present on the covered skin or mucous membrane can multiply at enhanced rates, particularly when the environment is moist and/or not exposed to air. Secretions from these microbes include acid or alkali excretions which can alter the pH of the skin, toxin secretion and enzyme secretion, including protease secretion. These secretions and excretions can cause skin and mucous membrane irritation, and in the more severe cases skin or mucous membrane breakdown, such as dermatitis.

Particular conditions which can occur following to the covering of skin or mucous membrane include thrush. Thrush is a fungal infection, by the Candida genus of yeast, particularly Candida albicans. Symptoms include itching, burning and soreness, and inflammation of the infected area. The wearing of sanitary towels, incontinence pads, nappies and/or tight underwear can produce conditions favourable to Candida growth, which can lead to thrush. The coatings of the present invention may be effective against fungi such as yeast, and accordingly it will be understood that providing the coatings of the invention on the above mentioned items may enable the treatment and/or prophylaxis of thrush.

Similarly, contact dermatitis (commonly known as nappy rash) may be caused by the wearing of incontinence pads or nappies. Damp or wet skin loses its structure, high pH can promote bacterial growth and the bacteria can secrete enzymes which break down the skin tissue. This environment can also promote or exacerbate pressure ulcers (commonly known as bed sores), which are particularly problematic when they become infected. The coatings of the present invention have been found to be effective against bacteria, and accordingly it will be understood that providing the coatings of the invention on tampons, sanitary towels, incontinence pads or nappies may enable the treatment and/or prophylaxis of contact dermatitis and/or pressure ulcers.

For similar reasons, contact dermatitis and yeast infections can occur under medical dressings, for example dressings for wounds and burns. An additional consideration with medical dressings is the need to prevent bacterial infection of the wound or burn. When skin is burnt, a large amount of tissue may be damaged which can reduce or destroy the natural barrier properties of skin, and wounds which break the skin also affect the barrier properties of skin. This can lead to opportunistic infection that can delay healing, and to septic shock. Additionally, microbial infection, particularly bacterial infection, can be a problem after surgery. The use of medical or surgical devices, for example implantable medical devices, which are coated with the present antimicrobial coatings may help to prevent or treat post-surgical infection. Accordingly, it will be understood that providing the coatings of the invention on dressings for wounds and/or burns may enable the treatment and/or prophylaxis of contact dermatitis and/or microbial infection.

The copper oxo-hydroxide nanoparticle compositions of the present invention, then, can be used in the manufacture of a medicament for the treatment and/or prophylaxis of microbial infection, and/or of skin or mucous membrane disorders such as inflammation and dermatitis. In particular, the antibacterial or antimicrobial coatings may be useful for the treatment and/or prophylaxis of infection of a wound, infection of a burn, infection of a pressure ulcer, post-surgical infection, thrush, contact dermatitis and pressure ulcers. The microbial infection may be by any microbe, in particular bacteria and/or yeast such as Staphylococcus sp., such as S. aureus, Pseudomonas sp., such as P. aeruginosa, Micrococcus sp., such as M. luteus, Saccharomyces sp., such as S. cerevisiae, Candida sp., such as C. albicans, Staphylococcus sp., such as S. epidermis, Streptococcus sp., such as S. pyrogenes, Klebsiella sp. and Escherichia sp., such as E. coli, Chlamydia sp. The compositions may further be active against viruses or parasites.

The medicament may be a substrate coated by the coating methods of the present invention. For example, then, the medicament may be a coated substrate such as a coated medical device, for example an implantable medical device. Examples include a surgical seed, catheter (such as a urinary catheter, a vascular access catheter, an epidural catheter), a vascular access port, an intravascular sensor, a tracheotomy tube, a percutaneous endoscopic gastrostomy tube, an endotracheal tube, an implantable prosthetic device, such as a stent and related short-indwelling or biocontacting devices. The medicament may be a coated substrate such as a coated nappy, sanitary towel, tampon, incontinence pad, dressing such as a wound or burn dressing, bandages or underwear. Many of these substrates (particularly nappies, sanitary towels, incontinence pads and dressings such as wound or burn dressings) comprise a non-woven fabric component, which may be in contact with skin or mucous membrane in normal use. The present inventors have demonstrated that the coatings and coating methods of the present invention are particularly suited to non-woven fabric substrates.

As used herein, the term “non-woven fabric” includes fabrics or textiles formed from a web of fibres. In non-woven fabric, the fibres are not woven or knitted. Non-wovens are typically manufactured by putting small fibers together in the form of a sheet or web, and then binding them mechanically. Example non-woven fabrics include polypropylene non-wovens.

It will be understood that the manufacturing process of the medicament may include providing an antimicrobial coating on a substrate. Accordingly, the manufacture of the medicament may comprise any of the steps of the methods described herein for providing antimicrobial coatings.

The present invention also provides substrates coated by the present methods. The coated substrates may be for use in a method of medical treatment, and include the coated substrates mentioned above as possible medicaments. It will be understood that the present invention also provides a method of medical treatment for the treatment and/or prophylaxis of microbial infection and/or of disorders of the skin or mucous membrane, and the use of the present coated substrates in such methods. The coating methods of the present invention are applicable to coating the substrates mentioned herein, as medicaments or otherwise.

As well as the applications described above, the antimicrobial coatings may also be provided on other equipment for use in medical applications, for example in hospitals. There is significant interest in controlling infection in hospitals, in particular bacterial infection such as MRSA and Clostridium difficile. As discussed above, microbial colonisation of surfaces is a particular problem. However, the present coatings have been found to be effective against many species of microbe, and so it will be understood that providing the present antimicrobial coatings on the surface of hospital equipment may be beneficial. Accordingly, substrates which may be coated according to the present invention include medical equipment and devices which contact the body or body fluids in normal use. For example, suitable substrates include tubes, fluid bags, catheters, syringes and surgical equipment such as scalpels and forceps etc. Additionally, other equipment, for example equipment used in hospitals (e.g. healthcare equipment) may be coated according to the present invention, for example gowns (e.g. surgical gowns), surgical masks, protective gloves (e.g. surgical and examination gloves), curtains, uniforms and bedding such as pillow cases, waterproof mattress covers (for example in babies cots and intensive care beds) and sheets.

Alternative healthcare equipment includes surgical draperies, surgical socks, furniture such as tables including bedside tables, beds, and seating surfaces, and other equipment including storage containers, filters, and service trays.

Additionally, the coatings of the invention are useful in coating equipment which it is desirable to keep free of microbes, for example equipment which is used in processing of food, for example kitchen equipment and surfaces, and factory equipment used in the manufacture or processing of food. For example, substrates which can be coated according to the present invention include containers (such as food storage containers), conveyors, blades, mixers, rollers and kitchen utensils (such as cutting and serving implements). Additional substrates include food preparation surfaces, flexible and rigid packaging and door handles.

Additionally, protective clothing worn by workers, for example overalls, gloves, masks and hats could be coated. Other clothing which may be coated includes undergarments, socks, athletic apparel, surgical apparel, healthcare apparel, shoes and boots.

Other substrates suitable for coating include filters, for example medical filters (including respirator filtration media and fluid filtration media), and other filters including HVAC filtration media, water filtration media and fluid filtration media.

Further suitable substrates include currency, debit/credit cards, industrial waste and water handling equipment, petrochemical and crude oil production, distribution and storage equipment and infrastructure. Additional suitable substrates include personal protective equipment and military apparatus such as face masks, respirators, decontamination suits and gloves.

### EXPERIMENTAL EXAMPLES

All experiments were carried out using ultra high pure (UHP) water (distilled deionised water; 18.2 ΩM/cm), and at room temperature (20±2° C.), unless otherwise stated.

1.1 Synthesis and Preparation of Copper Materials

1.1.1 Copper Chloride Stock Solution

CuCl₂.2H₂O was dissolved in UHP water to produce a concentrated stock at 40 mM (2542 ppm Cu), which was used in subsequent assays.

1.1.2 CuO Nanoparticles

Commercial CuO nanoparticles were obtained from Sigma-Aldrich (544868) and used as received and used for comparison with the antibacterial compositions of the present invention. Stock suspensions were prepared by dispersing the nanopowders in UHP water at 20 mM (1270 ppm Cu).

1.1.3 Silicate-Stabilised Copper Hydroxide Nanoparticles (CuSi NPs)

CuSi nanoparticles were prepared for comparative purposes by mixing a 400 mM sodium silicate solution at ca. pH 12, with a copper chloride solution (40 mM Cu), in a volume ratio of 1:1. The resulting suspension, containing 20 mM Cu and 200 mM Si, was pH adjusted to 12±0.2 with 5M NaOH, and was kept under stirring for 24 hours. After this period a light blue clear solution had been formed.

1.1.4 Cu-EDTA Complexes

Cu-EDTA complexes were freshly prepared by dissolving CuCl₂.2H₂O and disodium ethylenediaminetetraacetate (EDTA) di-hydrate in UHP water. The pH was adjusted to 7.5±0.2 with 1M NaOH. Various Cu:EDTA ratios were achieved by maintaining concentration copper at 20 mM (ca. 1270 ppm), whilst changing that of EDTA—20, 100 and 200 mM—thus achieving Cu-EDTA ratios of 1:1, 1:5 and 1:10, respectively. Copper solubility was confirmed by ICP-OES using elemental phase distribution (see 2.4.1.).

1.1.5 Tartrate Adipate Modified Copper Oxo-Hydroxide Nanoparticles (CuTartAd NPs)

An acidic solution comprising 40 mM copper chloride, 20 mM adipic acid and 20 mM tartaric acid was prepared. The pH of this, initially acidic, solution was raised through drop-wise addition of 5M NaOH up to pH 8.2±0.2. The final suspension contained ca. 40 mM (2500 ppm) Cu.

Nanoparticles synthesised as per Example 1.1.5 were characterised for copper phase distribution. During the synthetic process, soluble copper converted to particulate copper oxo-hydroxide as pH increased. Above pH 5, the particulate phase was mostly composed of nanoparticles (fraction greater than 80% of total particulate).

1.2 Nanoparticle Characterisation

1.2.1 Hydrodynamic Particle Size Distribution

Hydrodynamic particle size distribution of nanoparticles was determined by Dynamic Light Scattering (DLS) on a Zetasizer NanoZS (Malvern Instruments). In a typical experiment, 0.5 to 1 ml of nanoparticulate suspension (as prepared in 2.1.) was transferred into a small disposable cuvette at room temperature (20±2° C.) and 3 measurements were carried out using the following settings:

| | |
|---|---|
| Material Refractive Index | 0.192 |
| Absorption | 0.1 |
| Dispersant Refractive Index | 1.330 |
| Viscosity | 1.00331 mPa · s |

1.2.2 Zeta Potential

Zeta potential was analysed on a Zetasizer NanoZS (Malvern Instruments), by Laser Doppler Micro-electrophoresis, using a dielectric constant of 78.5 and a viscosity of 0.89 cP. Nanoparticle suspensions at ca. 1270 ppm Cu were transferred into clear disposable zeta cells to perform the measurement.

1.2.3 Transmission Electron Microscopy (TEM)

A suspension of CuTartAd NPs containing 2500 ppm Cu was analysed by TEM. TEM grids were prepared by dispersing the nanoparticulate suspension in methanol and drop-casted on holey carbon film TEM grids (Agar Scientific). Images were obtained on a CM200 (S)TEM fitted with an Oxford Instruments X-Max 80 mm2 SD detector and AZTEC analysis software.

1.2.4 X-Ray Diffraction (XRD) Analysis

CuTartAd NPs were dried at 45° C. for 24 hours and manually milled prior to conventional X-Ray Diffraction (XRD) analysis.

1.3 Bacterial Work

1.3.1 Heavy Metal MOPS (HMM) Medium, pH 7.2±0.2.

HMM is a copper-free defined medium developed for testing heavy metals and here was supplemented with glucose and cas-amino acids (acid hydrolysate of casein) to provide all basic nutrients required for bacterial growth. HMM was prepared from concentrated stock solutions of each reagent, and pH adjusted to 7.2±0.2 (Table 1). Freshly prepared medium was immediately autoclaved at 121° C. for 15 minutes, let cool down and stored at 4±2° C. Autoclaved medium was used within a month from preparation.

TABLE 1
Composition of HMM medium.

| | Concentration | |
|---|---|---|
| Reagent | in HMM medium | |
| 3-(N-morpholino)propanesulfonic | 40 | mM |
| acid (MOPS) | | |
| KCl | 50 | mM |
| NH₄Cl | 10 | mM |
| MgSO₄ | 0.5 | mM |
| FeCl₃•6H₂O | 1 | μM |
| Glycerol-2-Phosphate | 1 | mM |
| Glucose | 0.4% | (w/v) |
| Casein acid hydrolysate | 0.1% | (w/v) |

1.3.2 Bacterial Growth Inhibition Assay

Antimicrobial activity was assessed through determination of bacterial growth inhibition in the presence of copper compounds. A turbidimetric assay was used to follow bacterial concentration over time as this is proportional to optical density (OD at 595 nm) in liquid medium, allowing an easy screening of bacterial growth over time. Escherichia coli NCTC11100 and Staphylococcus aureus RN4220 were the tested microorganisms in this assay. Stock bacterial colonies were kept in cultivated in agar plates and on the day before the experiment, one colony was transferred into 10 ml of HMM liquid medium and grown overnight at 30° C. under constant shaking (80 rpm) in an incubator. On the day of the assay, the OD of the bacterial suspension was measured at 595 nm on a plate reader (Multiskan RC 351, Labsystems) and diluted in HMM to achieve an OD between 0.05 and 0.10 (CFU), ensuring that the initial concentration of bacteria was kept constant throughout the assays. Copper stock solutions (refer to section 2.1) were sequentially diluted in HMM to achieve typical concentrations between 0.8 and 100 ppm Cu in a volume of 0.1 ml. Next, 0.1 ml of bacterial culture was added and incubated with copper at 30° C. under constant agitation (80 rpm). Final copper concentrations in the assay ranged between 0.4 and 50 ppm, and OD was measured every hour for a typical period of 7-8 hours to follow bacterial growth. OD background, i.e. OD absorbance not caused by bacteria, was determined to remove readout interference from copper and broth. Growth inhibition was calculated as follows:

$$Growth Inhibition \% = ( \frac{OD Control - OD Copper}{OD Control} ) \times 100$$

OD control: OD of bacteria incubated in HMM in the absence of copper, after subtraction of OD of medium (no bacteria).

OD copper: OD of bacteria incubated in HMM in the presence of copper, after subtraction of OD of medium plus a matching concentration of copper (no bacteria).

1.3.3 Copper-Bacteria Association

The association of copper with E. coli NCTC11100 cultures was studied by initially growing the bacterial cultures overnight in HMM at pH 7.2±0.2 (as described in 2.3.2), to reach the stationary phase, such that bacterial concentrations remained constant throughout the assay. This concentration was determined to be 9×108 CFU/ml by agar plate counting. Next, copper chloride stock solution was diluted in the bacterial cultures to 3 and 12.5 ppm Cu, and incubated at 30° C. Samples were collected at 0, 2, 4 and 8 hours. At each time point, one aliquot of each sample was used to determine total copper concentration, and a second one was centrifuged for 5 min at 16000 g on a benchtop Biofuge Pico (Heraeus), to sediment bacteria and associated copper. Free copper in the supernatant was analysed by inductively coupled plasma-optical emission spectroscopy (as in 2.4.1.). Lastly, copper associated to bacteria was determined as below:

[Cu] associated to bacteria=Total [Cu]−Supernatant [Cu]

1.4 Chemical Assays

1.4.1 Elemental Copper Analysis

Inductively coupled plasma-optical emission spectroscopy (ICP-OES) was used to determine elemental copper concentration. All samples were diluted in 5% HNO3 (v/v) at least 24 hours prior to analysis to dissolve copper materials. Calibration standards were matrix-matched in 5% HNO₃, ranging from 0.1 to 100 ppm. The line used for copper detection was 324.754 nm.

1.4.2 Determination of Copper Phased Distribution: Soluble, Nanoparticulate and Microparticulate Fractions

Phase distribution was determined by separating soluble (<1.4 nm), nanoparticulate (<100 nm) and submicron/microparticulate (>100 nm) copper. Three samples were collected, 1) Total, analysed neatly for copper concentration; 2) supernatant, centrifuged for 5 minutes at 16000 g, followed by supernatant analysis; and 3) soluble, filtered through a 3 KDa filter. Phase distribution was then calculated as:

$$Copper Microparticulate \% = \frac{{Cu}_{Total} - {Cu}_{Supernatant}}{{Cu}_{Total}} \times 100$$

$$Copper Soluble \% = \frac{{Cu}_{Soluble}}{{Cu}_{Total}} \times 100$$

$$Copper Nanoparticulate \% = {Cu}_{Soluble} ( \% ) - {Cu}_{Microparticulate} ( \% )$$

Copper-Based Nanoparticles Dissolution

Dissolution of copper nanoparticles was studied in bacterial growth medium (HMM). Copper materials were diluted from stocks (see section 2.1) to 12.5, 25 and 50 ppm copper, and aliquots were collected at 0, 2, 4 and 8 hours. Fraction of soluble copper was determined by analysis of phase distribution as described in described in section 2.4.2.

1.4.4 Copper Dispersibility

Stock copper materials were diluted in 50 mM 3-(N-morpholino)propanesulfonic acid (MOPS) buffer at pH 7.4±0.2. Final copper concentrations varied between 10 and 500 ppm. Microparticulate copper was removed by centrifugation at 16000 g for 5 minutes. Total copper and supernatant (i.e. disperse fraction) were analysed by Inductively Coupled Plasma-Optical Emission Spectroscopy (ICP-OES) as described in section 2.4.1.

1.4.5 Copper Release from a Gel Matrix—Gel Release Assay

CuCl₂ and CuTartAd nanoparticles, at pH between 7 and 8, were incorporated into a hydroxyethylcellulose (HEC) matrix by diluting the original stocks containing ca. 2500 ppm, down to 250 ppm in UHP water, and next dissolving hydroxyethylcellulose (HEC) to achieve a concentration of 2% (w/v). The mixture was kept under moderate stirring in a rotary shaker until a homogeneous gel was formed. 10 g of the gel was poured into a 50 ml falcon tube and let settle down overnight. 10 ml of a 50 mM NaHCO3 solution at pH 7±0.2 were carefully transferred on the surface of the gel. Samples were collected over 24 hours and copper concentration in the overhead solution was determined by ICP-OES (see section 2.4.1.).

### 2. COPPER-BASED NANOPARTICLES AS DELIVERY AGENT FOR BIOCIDAL COPPER IONS

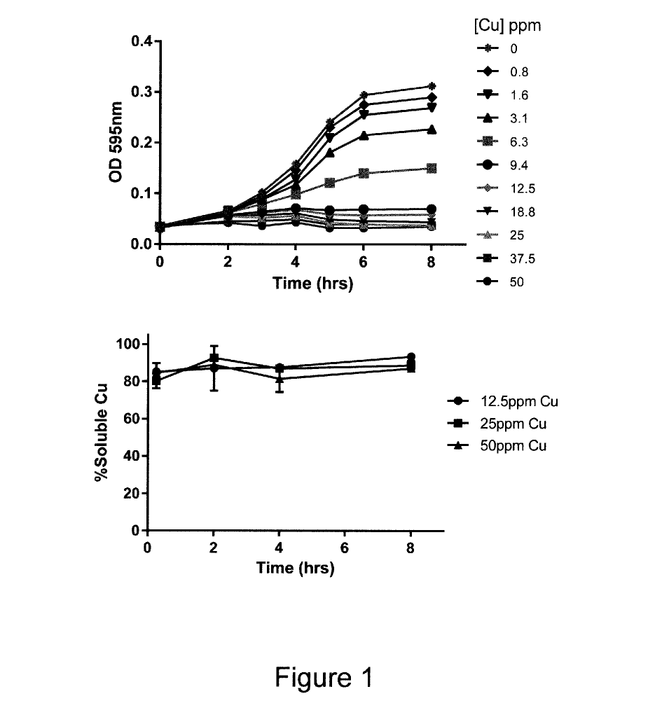

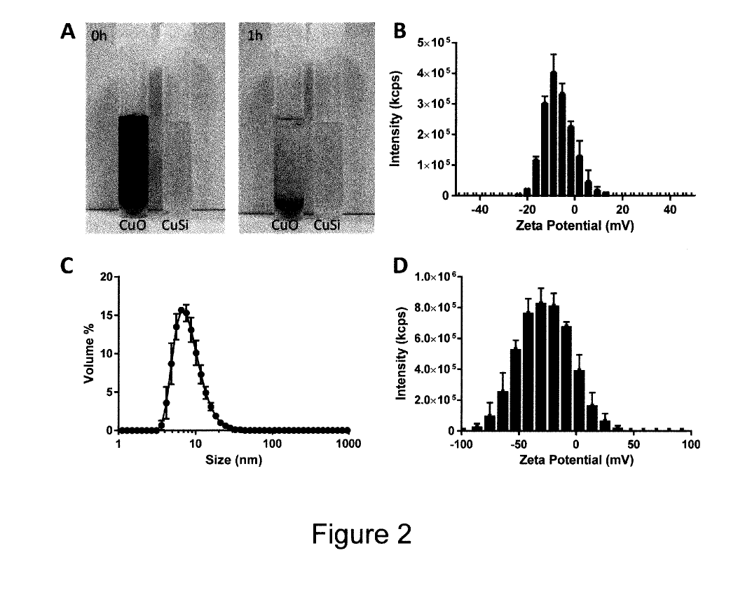

A turbidimetric assay was developed to test antimicrobial activities of nanoparticulate materials in which E. coli concentration was followed through optical density measurements in a liquid media that enabled in situ characterisation of copper phase distribution. Bacteria were incubated in the presence of a broad range of copper concentrations, and copper chloride was used as a reference biocidal material to provide soluble copper, see FIG. 1. The next stage of this work required the selection of appropriate nanoparticulate materials. Initially, commercial CuO nanoparticles with indicated sizes of ca. 50 nm were studied; however, when dispersed in water these nanoparticles formed large micron-sized agglomerates (FIG. 2A). Their unexpected lack of dispersibility was explained by weak surface repulsion as evidenced by a zeta potential peak of only −7.1±0.5 mV.

Consequently, alternative copper-based nanoparticles were developed to act as genuine nanoparticulate agents, i.e. stable colloids at concentrations suitable for delivery in the assay. Silicate was chosen as stabiliser given its lack of antimicrobial activity, such that particle toxicity would be driven by copper alone. The resulting reference silicate-stabilised copper hydroxide nanoparticles (CuSi NPs) were thus synthesised through co-precipitation, in which a copper chloride solution was mixed with alkaline sodium silicate solution at ca. pH 12. Initially, Cu²⁺ ions precipitated as Cu(OH)₂, forming micron-sized agglomerates, but these dispersed over time to form small nanoparticles with a hydrodynamic diameter peak of 8.5±0.3 nm (FIG. 2D). This process of dispersion was anticipated to be driven by the adsorption of negatively-charged silicate ions to copper hydroxide agglomerates. Additionally, high ratios of silicate-to-copper (10:1) ensured efficient negative charge repulsion, as confirmed by the Zeta Potential measurement, which showed that the nanoparticles were sufficiently negatively charged (−31±9 mV) to resist agglomeration (FIG. 2B).

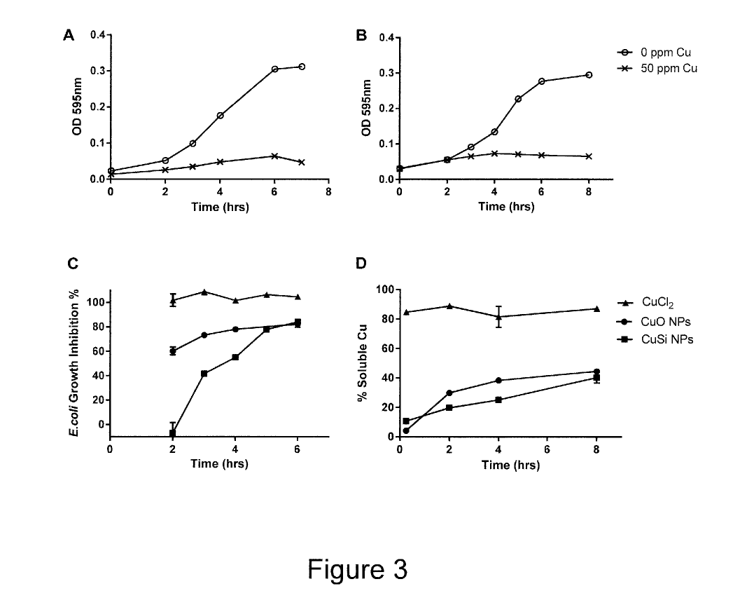

Next, the antimicrobial activities of these two distinct types of nanoparticle, CuO and CuSi, were compared to a soluble copper control (CuCl2) across a range of copper concentrations (0.8-50 ppm Cu) against E. coli. Nanoparticles were found to be less efficacious than soluble copper, which is particularly well illustrated at an exposure level of 50 ppm Cu (FIG. 3). Here, growth inhibition upon exposure to CuSiNPs was more pronounced at the latter time points implying a gradual and delayed effect of CuSi NPs on bacteria. Surprisingly, despite the observed agglomeration, CuO nanoparticles showed equivalent growth inhibition to CuSi NPs at 6 hours. However, the increase in inhibition over time was not as pronounced as for CuSi NPs. Similar biocidal kinetics of nanoparticles, i.e. both showed gradual and delayed growth inhibition, may imply a common nanoparticulate mode of action distinct from that of soluble copper, e.g. adhesion to bacterial membrane. However, such a mechanism seems unlikely given the disparities in physicochemistry between the nanoparticles (e.g. size, charge and composition). For instance, nanoparticles with different surface charge would be expected to show different affinities for the bacterial membrane, and consequently different antimicrobial activities, which were not observed here. More plausibly, a dissolution mediated mechanism based upon the release of copper ions from nanoparticles would explain delayed toxicity relative to copper chloride, and is supported by respective dissolution profiles of both materials. Release of copper ions accorded to growth inhibition, in which faster dissolution of CuO NPs resulted in greater antimicrobial efficacy at earlier time points (2-4 hours). After 4 hours, the percentage of soluble copper released from either of the nanoparticles was equivalent (ca. 40% of total copper) which translated into similar bacterial growth inhibition (ca. 80%). Similarity in dissolution profiles of the two types of nanoparticles was surprising, since smaller CuSi NPs were expected to dissolve faster, due to their higher surface area-to-volume ratio, but the presence of insoluble silicates in the structure of nanoparticles may have retarded dissolution. Critically, the association between dissolution and bacterial growth inhibition implied that biocidal activity was driven by soluble copper for both types of nanoparticles.

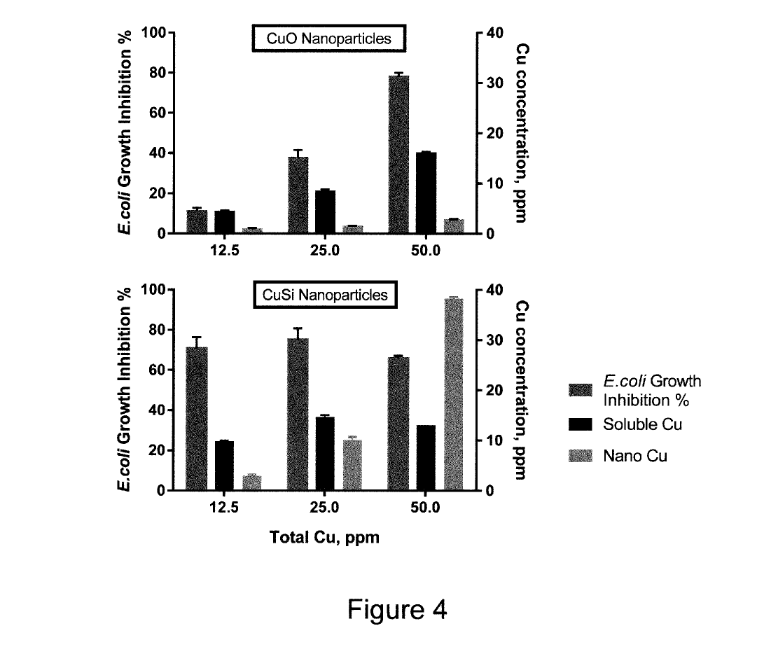

To further clarify the ‘dissolution’ theory, E. coli growth inhibition was compared for soluble (<1.1 nm) as well as nanoparticulate (1-100 nm) copper fraction in the bacterial culture medium. A dose response was observed for CuO materials, in which increasing levels of copper (12.5, 25 and 50 ppm) led to an increase in growth inhibition (FIG. 4). However, agglomeration resulted in very low concentrations of nanoparticulate copper (<3 ppm), and thus the increase in growth inhibition could not be attributed to this fraction, but rather to the increase in soluble copper. CuSi NPs behaved in a distinct manner to commercial CuO NPs: here, greater quantities of material resulted in increased nanoparticulate copper concentrations (3, 10 and 38 ppm) but relatively unchanged levels of soluble copper (10-15 ppm). However, such increase in nanoparticulate copper did not result in additional biocidal action and, instead, growth inhibition accorded with relatively static levels of soluble copper.

The dependence of biocidal activity upon soluble copper, suggests that optimal antibacterial efficacy would be achieved by copper salts, which readily delivered soluble copper ions in the medium used for the growth inhibition assay (FIG. 4). However, use of copper as an antimicrobial agent in clinical applications, such as wound healing (including treatment of cuts and abrasions), requires formulations at concentrations much greater than those tested in the antimicrobial assay (<50 ppm) to enable the delivery of quantities of copper that are effective at killing bacteria. In addition, these formulations should be delivered at physiological pHs (pH 6-8) to avoid further detrimental effects of extreme pHs on a skin that is already vulnerable by the wound. Therefore, appropriateness of copper salts in formulations for wound healing was investigated by quantifying their dispersibility, i.e. non-precipitated, in a MOPS buffer at pH 7.4±0.2, as an indication of bioavailability. Most copper precipitated as large centrifugible agglomerates, reducing the dispersible fraction to a maximum of ca. 10 ppm of Cu, despite addition of copper at concentrations as high as 500 ppm.

Therefore, copper salts have limited used in formulations as they are not stable towards precipitation which limits the bioavailable copper.

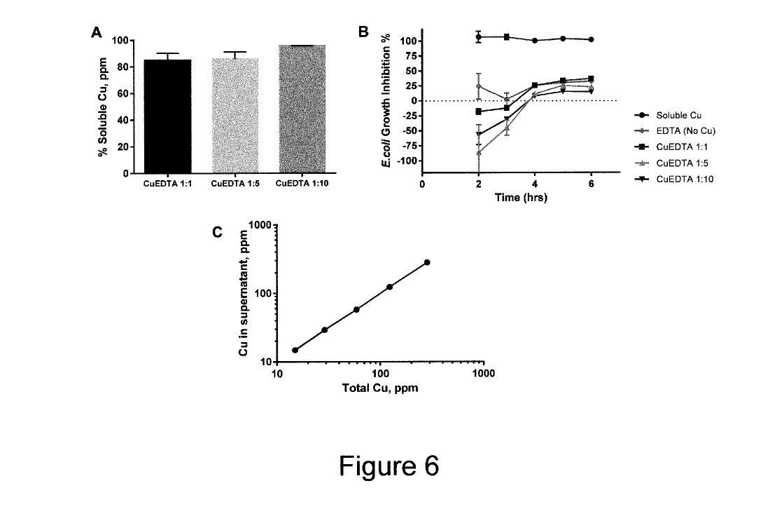

The undesirable precipitation of copper salts at physiological pH occurred through the formation of large (centrifugible) copper hydroxide agglomerates that can be prevented through the use of complexing agents (e.g. EDTA). For instance, in the growth inhibition assay, copper was maintained soluble in the bacterial medium by complexing agents present in the medium, possibly amino acids. The viability of this strategy was employed at various Cu:EDTA ratios, and the resulting solutions were tested in the bacterial assay (FIG. 6A). Despite keeping copper in solution, Cu-EDTA complexes showed modest growth inhibition (<40%; FIG. 6B). Interestingly, EDTA alone had antibacterial effect, which arguably could be responsible for most the effect of the complexes. These observations showed that whilst the release of copper ions is essential for antibacterial purposes, the form of soluble copper is also important, and when in the presence of strong complexing agents, such as EDTA, availability of free copper ions is reduced since such strong chelates ‘compete’ with bacteria for copper, resulting in reduced toxicity.

Despite the delayed growth inhibition observed for copper-based nanoparticles in comparison to copper salts (FIG. 3), nanoparticles showed a greater antimicrobial activity than copper complexes, implying a greater capacity to deliver free copper ions. Therefore, their appropriateness for clinical formulations was also tested in the same conditions as described for copper chloride.

In summary, this initial body of work demonstrated that copper-based nanoparticles had little or no direct effect on bacteria, and their biocidal activity was triggered via the release of copper ions, the main biocidal form of copper. Unlike copper salts and copper complexes, which are not appropriate for the release of free copper ions, nanoparticles have great potential as a delivery systems for such species and can remain disperse at suitable concentrations for formulation in clinical applications. However, the CuO and CuSi nanoparticles tested in this work are not optimal as the rate of copper released was found to be low.

### 3. NOVEL EFFECTIVE COPPER-BASED NANOPARTICLES FOR THE DELIVERY OF COPPER IONS

3.1 Introduction

As maximum antimicrobial efficacy of copper-based nanoparticles can be achieved through rapid release of copper ions, we attempted to produce such labile materials by modifying their mineral structure via synthetic methodologies that promote the formation of unstable mineral phases.

3.2 Ligand Modified Copper Oxo-Hydroxide Nanoparticles

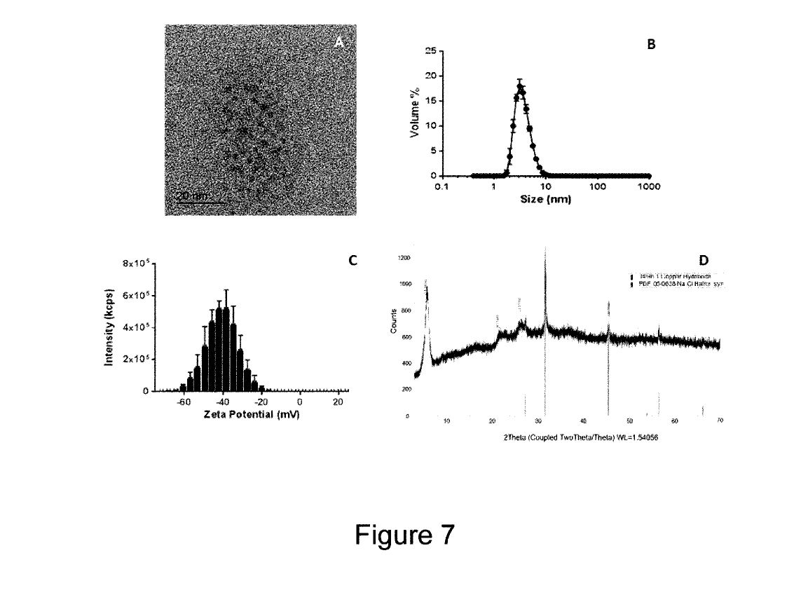

Copper oxo-hydroxide minerals were prepared through pH-driven precipitation of a copper chloride solution by drop-wise addition of sodium hydroxide, which forced the conversion of copper ions to copper oxo-hydroxides. This was carried out in the presence of carboxylate ligands, namely tartaric acid and adipic acid, which controlled mineral growth at the nanoscale as a result of ligand incorporation and surface capping of the mineral growth front, to produce small and stable nanoparticles, with core structures of 2 to 5 nm (FIG. 7). Tartaric acid played a key role in stabilising the nanoparticles in solution via electrostatic repulsion, presumably through its negative carboxylate groups—deprotonated above pH 4.4, its second pKa. Zeta potential showed that nanoparticles were sufficiently negatively-charged (peak at −39 mV) to prevent particle aggregation due to strong particle repulsion (|Zeta Potential|>30 mV), and therefore the formation of a very stable suspension. XRD analysis indicated an amorphous mineral phase, which is likely due to the incorporation of tartaric acid in the mineral structure. In contrast, the present inventors believe that adipic acid, a weak ligand with low affinity for copper, was mainly used for its buffering capacity to control the pH during the synthesis. The CuTartAd NPs also showed an amorphous mineral phase, likely due to surface disruption of the mineral lattice by tartaric acid. Amorphousness may impact on lability, since materials with amorphous minerals phase are more labile than crystalline ones.

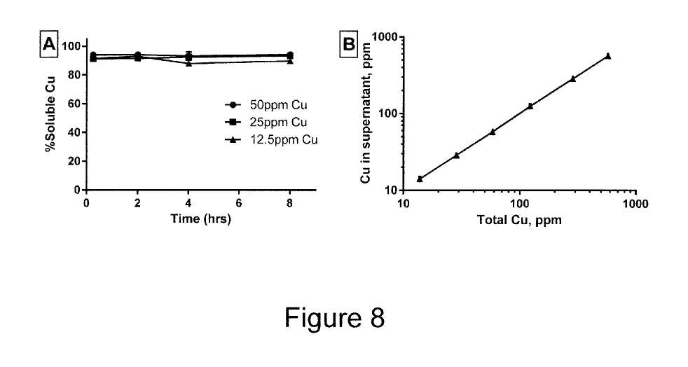

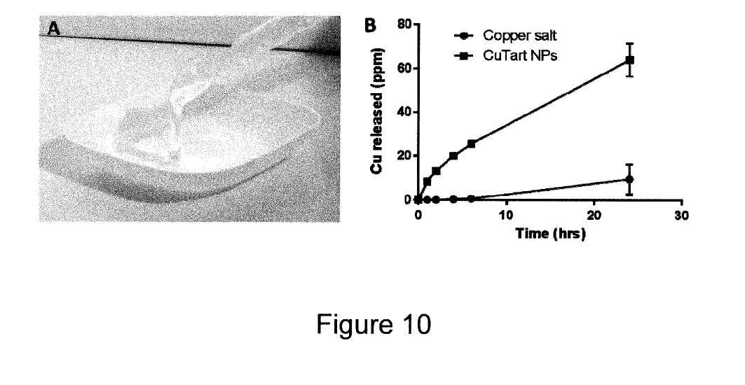

Following synthesis of CuTartAd nanoparticles, their dissolution profile was determined in bacterial growth medium upon dilution to 12.5, 25 and 50 ppm Cu, concentrations normally used in the antimicrobial assays. Nanoparticles dissolved immediately after dilution in the medium (FIG. 8), and remained in solution for at least 8 hours, the period studied in this assay. As previously observed for CuSi NPs, CuTartAd NPs were stable in dispersion at high copper concentrations (FIG. 10B), but unlike CuSi NPs, were extremely labile, demonstrating rapid release of copper in bacterial growth medium.

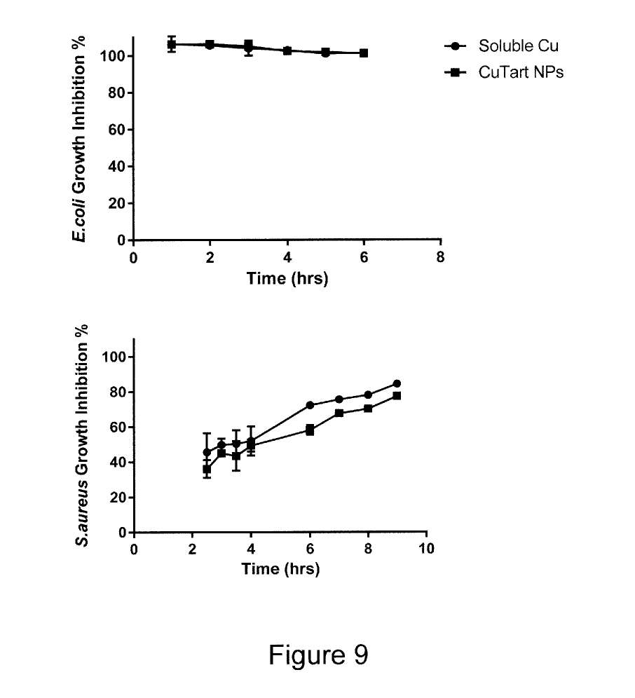

Having confirmed lability, antimicrobial efficacy testing of CuTartAd NPs ensued. Two standard bacterial models were used to measure activity against both E. coli and S. aureus, a gram-negative and a gram-positive bacterium, respectively. CuTartAd NPs were found to be efficacious against both strains, inhibiting S. aureus growth by more than 80%, whilst fully inhibiting E. coli growth at incubations of 50 ppm Cu (FIG. 9A). This represented an improvement relative to CuSi NPs, which failed to fully inhibit E. coli growth at the same concentration (FIG. 3). These results reinforced the significance of soluble copper ions for antimicrobial effect; both nanoparticles, CuSi NPs and CuTartAd NPs, exhibited similar physicochemical properties (e.g. small size and negative charge), but different dissolution rates and corresponding differences in antibacterial activity. Moreover, CuTartAd NPs showed equal efficacy to soluble copper, demonstrating their suitability for delivery of biocidal copper. The ligand modified copper oxo-hydroxide nanoparticles of the present invention have a bactericidal effect against a broad range of microorganisms, including pathogenic models of P. aeruginosa and S. aureus (Table 3).

TABLE 3
Minimum bactericidal concentration (MBC) obtained from incubation
CuCl₂ or CuTartAd with several bacterial models, including
conventional lab strains (E. coli MC1061 and B. subtilis BR151),
pathogenic models (S. aureus RN4220 and P. aeruginosa) and ISO
standards for toxicity tests (V. fischeri). Each bacterial species
was incubated with Cu, both CuCl₂ and CuTartAd nanoparticles, in
liquid medium from 4 to 96 hours. Next, bacterial cultures were
transferred to agar plates and MBC values were determined through
visual inspection of colonies formed (n = 1).

| | MBC (ppm Cu) | | | | |
|---|---|---|---|---|---|
| | E. | B. | S. | P. | V. |
| Time (h) | coli | subtilis | aureus | aeruginosa | fischeri |
| CuCl₂ | | | | | |
| 4 | 100 | >100 | 100 | 100 | 100 |
| 24 | 50 | >100 | 50 | 100 | 100 |
| 48 | 10 | >100 | 50 | 100 | >100 |
| 72 | 50 | >100 | 50 | 100 | >100 |
| 96 | 50 | >100 | 50 | 100 | >100 |
| CuTartAd | | | | | |
| 4 | 100 | >100 | 100 | 100 | 100 |
| 24 | 100 | >100 | 50 | 100 | 100 |
| 48 | 10 | >100 | 50 | 100 | >100 |
| 72 | 50 | >100 | 50 | 100 | >100 |
| 96 | 10 | >100 | 50 | 100 | >100 |

Broad-spectrum antimicrobial efficacy across gram negative and gram positive species is advantageous for numerous clinical applications, in particular to combat wound infections—due to the variety and number of pathogens wounded skin is exposed to, and the deleterious impacts of infection upon healing. Thus, having demonstrated the biocidal efficacy of CuTartAd NPs, their appropriateness for topical delivery was tested. Typical formulations for wound healing comprise dressings or creams in which actives are impregnated and then released upon exposure to moisture. Here, as a proof of principle, nanoparticles were incorporated in a hydroxyethylcellulose (HEC) matrix. HEC is a cellulose derivative that has been widely used in health care products and cosmetics, and unlike dressings or other matrices (e.g. polyethylene glycol), HEC does not require any further processing (e.g. heating and drying) of nanoparticles during matrix preparation, with minimal alterations to their physicochemical properties. Thus, incorporation of CuTartAd NPs was achieved simply by diluting colloids to the desired concentration and dissolving HEC into the suspension which resulted in the formation of a homogeneous gel that embedded the nanoparticles.

Importantly, copper was released from this gel: over 24 hours, HEC matrices impregnated with CuTartAd NPs at 250 ppm were found to release 64±8 ppm—a concentration more than sufficient to inhibit bacterial growth. In contrast, gels formulated with 250 ppm Cu chloride failed to release more than 10 ppm Cu over the same time period. This confirmed the inappropriateness of copper salts as delivery agents at physiological pHs. As such, CuTartAd nanoparticles were here shown to have suitable properties for the combat of wound infections, as they released biocidal copper ions readily, which resulted in high antimicrobial activity and were appropriate for topical delivery.

### 4. SYNTHETIC EXAMPLES

4.1 CuOH 40 Tart20 Ad20 Nanoparticles Prepared from CuSO₄

Nanoparticles were synthesised as per Example 1.1.5, but CuSO₄ was used instead of CuCl₂. Nanoparticles synthesised from CuSO₄ as per this example were characterised for copper phase distribution. During the synthetic process soluble copper converted to particulate copper oxo-hydroxide as pH increased. By pH 7, the particulate phase was mostly composed of nanoparticles (approximately 80% of total copper). Hydrodynamic particle size was determined by Dynamic Light Scattering during the synthetic process of tartrate-adipate modified copper oxo-hydroxide nanoparticles synthesised as per Example 1.1.5. In addition to increased dispersibility, pH increase resulted in reduced particle size. For instance nanoparticles recovered at pH 6.5 exhibit larger particle sizes (73±10 nm) than particles recovered at higher pHs (e.g. 4.6±0.5 nm at pH 8). When recovered at pH 8, these had hydrodynamic diameters between 1.5 and 20 nm, with mean diameters between 3 and 5 nm.

4.2 CuOH 40 Tart20 Ad20 Nanoparticles Prepared from CuNO₃

Nanoparticles were synthesised as per Example 1.1.5, but CuNO₃ was used instead of CuCl₂. Nanoparticles synthesised from CuNO₃ as per this example were characterised by Dynamic Light Scattering. When recovered at pH 8, these had hydrodynamic diameters between 2 and 10 nm, with mean diameters between 3 and 5 nm.

4.3 CuOH40 Gluconic Acid 60

Nanoparticles were synthesised as per Example 1.1.5., but gluconic acid (60 mM) was used instead of tartaric and adipic acids. Nanoparticles synthesised with gluconic acid as per this example were characterised for copper phase distribution. During the synthetic process soluble copper converted to particulate copper oxo-hydroxide as pH increased. By pH 6, the particulate phase was mostly composed of nanoparticles (fraction greater than 80% of total copper). Nanoparticles synthesised with gluconic acid as per this example were characterised by Dynamic Light Scattering. When recovered at pH 8, these had hydrodynamic diameters between 1 and 10 nm, with mean diameters between 2 and 4 nm.

4.4 CuOH 20 Glutathione 20

Nanoparticles were synthesised as per Example 1.1.5., but glutathione (20 mM) was used instead of tartaric and adipic acids. The initial concentration of CuCl₂ was also halved to 20 mM. Nanoparticles synthesised with glutathione as per this example were characterised for copper phase distribution. During the synthetic process soluble copper converted to particulate copper oxo-hydroxide as pH increased. Between pH 3 and 4, the particulate phase was mostly composed of large agglomerates (approximately 70% of total copper). By pH 6, these micron-sized particles dispersed and the particulate copper became mostly composed of nanoparticles (fraction greater than 80% of total copper). Nanoparticles synthesised with glutathione as per Example N4 were characterised by Dynamic Light Scattering. When recovered at pH 8, these had hydrodynamic diameters between 1 and 5 nm, with a mean diameter of approximately 2 nm.

4.5 CuOH 40 Tart 20 Ad20 Nanoparticles Prepared with Na₂CO₃

Nanoparticles were synthesised as per Example 1.1.5, but Na₂CO₃ was used instead of NaOH. Nanoparticles synthesised using acid Na₂CO₃ as the titrant as per this example were characterised for copper phase distribution. During the synthetic process soluble copper converted to particulate copper oxo-hydroxide as pH increased. By pH 7, the particulate phase was mostly composed of nanoparticles (fraction greater than 90% of total copper). Nanoparticles synthesised using acid Na₂CO₃ as the titrant (as per Example N5) were characterised by Dynamic Light Scattering. When recovered at pH 8, these had hydrodynamic diameters between 1 and 8 nm, with mean diameters between 2 and 4 nm.

4.6 Unmodified CuOH 40 (Comparative Example)

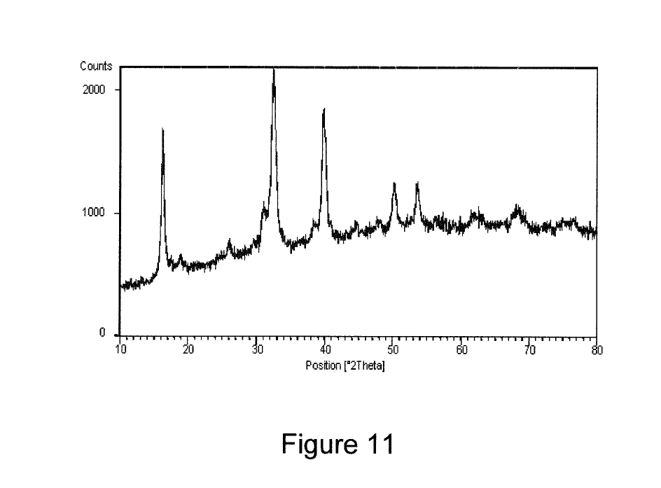

The same synthetic methodology described in the Example 1.1.5. was followed, but in the absence of tartaric and adipic acid. During the synthetic process of unmodified copper hydroxides, most soluble copper converted to particulate between pH 4.3 and 5.2. Above this pH, the particulate phase was entirely composed of large micron-sized particles (fraction greater than 95% of total copper). The XRD spectrum of the resulting material was also obtained (FIG. 11). The latter showed a crystalline pattern corresponding to paratacamite, a copper hydroxide of chemical formula Cu₂(OH)₃Cl in which a chlorine atom was incorporated in the mineral structure (bottom).

4.7 CuOH 40 Tart 20 Nanoparticles

Tartrate-modified copper oxo-hydroxide nanoparticles were synthesised as per Example 1.1.5., but in the absence of adipic acid. Nanoparticles synthesised in the absence of adipic acid as per this example were characterised by Dynamic Light Scattering. When recovered at pH 8, these had hydrodynamic diameters between 2 and 10 nm, with mean diameters between 3 and 5 nm.

4.8 CuOH 2000 Tart 1000 Nanoparticles

Tartrate-modified copper oxo-hydroxide nanoparticles were synthesised as per Example 4.7, but at higher concentration (2.0 M copper and 1.0 M tartaric acid). The resulting material was a viscous slurry.

4.9 Resuspension of CuOH 2000 Tart 1000 Nanoparticles

A slurry prepared as described in N9 was diluted to ˜50 mM in a 20 mM adipic acid solution Cu and the pH adjusted to 8 with NaOH. Nanoparticles synthesised from a concentrated slurry as per this example were characterised by Dynamic Light Scattering. When recovered at pH 8, these had hydrodynamic diameters between 2 and 10 nm, with mean diameters between 3 and 5 nm.

4.10 Removal of Unbound Ligands

Free ligand and salts were removed through a process of ethanolic precipitation, in which a suspension of CuTartAd NPs (synthesised as per Example 1.1.5.) was mixed with ethanol on a volume ratio of 1:2 nanoparticle suspension: ethanol. Next, the agglomerated nanoparticles were span down at 1500 rpm for 5 minutes and the supernatant (containing free ligands and salts) was discarded. The pellet, containing the nanoparticles, was resuspended to the original volume.

### 5. ACTIVITY ASSAY

5.1 Exposure of CuOH 40 Tart20 Ad20 Nanoparticles to Skin Fibroblasts

Human dermal fibroblast cells (cell line CCD-25SK) were incubated with CuTartAd NPs (0-200 ppm Cu) in Minimum Essential Medium (containing L-glutamine and Earle's salts) supplemented with 5% heat inactivated Fetal Bovine Serum, 1% Penicillin-Streptomycin, 1% Fungizone and 3.8% bovine serum albumin, at 37° C. under a humidified 5% CO₂ atmosphere for 48 hours. CuCl₂ and AgNO₃ were also tested in parallel as positive controls. Percentage of cell confluence was determined experimentally using an IncuCyte Zoom and plotted overtime to determine the area under the curve (AUC) for each concentration tested. Cell proliferation was used as an indication for cell toxicity and was determined by normalising the AUC of cells exposed to the testing compounds against those of cells growing at normal rates (control).

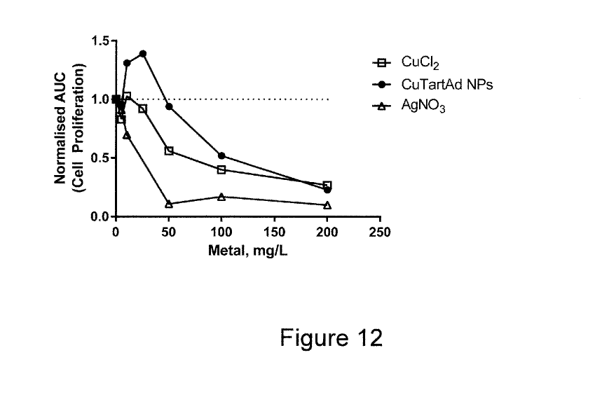

Skin Fibroblasts cells were exposed to CuCl₂, AgNO₃ or ligand-modified copper nanoparticles (synthesised as per Example 1.1.5) for 48 hours. As shown in FIG. 12, CuCl₂ and AgNO₃ caused a decrease in cell proliferation at lower concentrations (from 50 mg/L and 10 mg/L respectively) than with copper nanoparticles (from 100 mg/L). In addition to reduced toxicity, copper oxo-hydroxide nanoparticles promoted cell growth (increased cell proliferation) at low concentrations (10 and 25 mg/L Cu) indicating a beneficial effect on wound healing.

All publications, patent and patent applications cited herein or filed with this application, including references filed as part of an Information Disclosure Statement are incorporated by reference in their entirety.

1. An antibacterial composition comprising ligand-modified copper oxo-hydroxide nanoparticles, wherein the copper oxo-hydroxide nanoparticles have a structure in which the one or more ligands are non-stoichiometrically substituted for the oxo or hydroxy groups, wherein the one or more ligands comprise a carboxylic acid ligand, or an ionised form thereof.

2. The antibacterial composition comprising copper oxo-hydroxide nanoparticles according to claim 1, wherein the material has a polymeric structure in which the ligands are distributed within the solid phase structure of the copper oxo-hydroxide.

3. The antibacterial composition comprising copper oxo-hydroxide nanoparticles according to claim 1 or claim 2, wherein the ligand-modified copper oxo-hydroxide nanoparticles have one or more reproducible physicochemical properties.

4. The antibacterial composition comprising copper oxo-hydroxide nanoparticles according to any one of the preceding claims, wherein the one or more reproducible physicochemical properties are selected from dissolution profile, release of soluble copper as a percentage of total copper present in the composition and/or antibacterial activity as determined in a growth inhibition assay.

5. The antibacterial composition comprising copper oxo-hydroxide nanoparticles according to any one of the preceding claims, wherein the carboxylic acid ligand is a linear or cyclic mono or dicarboxylic acid ligand.

6. The antibacterial composition comprising copper oxo-hydroxide nanoparticles according to any one of the preceding claims, wherein the carboxylic acid ligand or the ionised form thereof is tartaric acid or tartarate, gluconic acid or gluconate, adipic acid or adipate, succinic acid or succinate, malic acid or malinate, glutaric acid or glutarate, pimelic acid or pimelate and/or glutathione, or wherein the carboxylic acid ligand is an amino acid or a sugar acid.

7. The antibacterial composition comprising copper oxo-hydroxide nanoparticles according to any one of the preceding claims, wherein the carboxylic acid ligand or the ionised form thereof is tartaric acid or tartarate or gluconic acid or gluconate or glutathione.

8. The antibacterial composition comprising copper oxo-hydroxide nanoparticles according to any one of the preceding claims, wherein the carboxylic acid ligand or the ionised form thereof is tartaric acid or tartarate in combination with adipic acid or adipate.

9. The antibacterial composition comprising copper oxo-hydroxide nanoparticles according to any one of the preceding claims, wherein:
(a) the nanoparticles in the composition have demonstrable M-L bonding as determined using infrared spectroscopy; and/or
(b) the nanoparticles in the composition are substantially amorphous as determined by XRD.

10. The antibacterial composition comprising copper oxo-hydroxide nanoparticles according to any one of the preceding claims, wherein the nanoparticles have a mean diameter between 1 and 100 nm, and optionally wherein the nanoparticles have a mean diameter between 1 and 10 nm.

11. The antibacterial composition comprising copper oxo-hydroxide nanoparticles according to any one of the preceding claims, wherein the composition is formulated at a pH between 6.0 and 8.0.

12. The antibacterial composition comprising copper oxo-hydroxide nanoparticles according to any one of the preceding claims, wherein the composition further comprises a matrix in which the nanoparticles are formulated.

13. The antibacterial composition comprising copper oxo-hydroxide nanoparticles according to claim 12, wherein the matrix comprises hydroxyethylcellulose or PEG.

14. An antibacterial composition comprising copper oxo-hydroxide nanoparticles according to any one of the preceding claims for use in a method for the treatment of wounds.

15. An antibacterial composition comprising copper oxo-hydroxide nanoparticles according to any one of the preceding claims for use in a method for the treatment or prevention of a microbial infection.

16. The antibacterial composition comprising copper oxo-hydroxide nanoparticles according to claim 15, wherein the microbial infection is a bacterial infection or a fungal infection.

17. The antibacterial composition comprising copper oxo-hydroxide nanoparticles according to any one of claims 14 to 16, wherein the composition is for treating a human or animal subject.

18. The antibacterial composition comprising copper oxo-hydroxide nanoparticles according to any one of claims 15 to 17, wherein the infection is caused by a gram-negative or a gram-positive bacterium.

19. The antibacterial composition comprising copper oxo-hydroxide nanoparticles according to any one of claims 15 to 18, wherein the bacterial infection is caused by an Escherichia sp., such as E. coli, a Staphylococcus sp., such as S. epidermis, S. aureus or meticillin-resistant staphylococcus aureus (“MRSA”), a Bacillus sp., such as B. subtilis, a Pseudomonas sp., such as P. aeruginosa, a Vibrio sp., such as V. fisheri, a Streptococcus sp., such as S. pyrogenes and S. pneumoniae, a Klebsiella sp., a Micrococcus sp., such as M. luteus, a Clostridium sp. such as C. difficile, an Acinetobacter sp. such as A. baumannii, a Mycobacterium sp., such as M. tuberculosis or a Salmonella sp. a Chlamydia sp. or a fungal species such as a Candida sp., such as C. albicans.

20. The antibacterial composition comprising copper oxo-hydroxide nanoparticles according to any one of claims 1 to 13, wherein the composition is for veterinary administration.

21. A pharmaceutical composition comprising the antibacterial composition comprising ligand-modified copper oxo-hydroxide nanoparticles according to any one of claims 1 to 13, and a pharmaceutically acceptable carrier.

22. The pharmaceutical composition of claim 21 wherein the composition is for topical delivery, vaginal delivery, nasal delivery, rectal delivery or oral delivery.

23. An article coated or treated with an antibacterial composition according to any one of claims 1 to 13.

24. The article according to claim 23, wherein the article is an implantable medical device or a coated substrate, such as a non-woven fabric substrate.

25. Use of copper oxo-hydroxide nanoparticle composition according to any one of claims 1 to 13 for the preparation of a medicament for the treatment or prevention of microbial infection.

26. A method of treating or preventing a microbial infection, the method comprising administering to a patient in need of treatment a therapeutically effective amount of copper oxo-hydroxide nanoparticle composition according to any one of claims 1 to 13.

27. The composition for use in a method of treatment, the use or the method according to any one of claims 1 to 13, wherein the composition is formulated for topical administration.

28. A process for producing a copper oxo-hydroxide nanoparticle composition according to any one of claims 1 to 13, the process comprising:
(a) mixing the solution comprising Cu²⁺ and a carboxylic acid ligand, and optionally one or more further ligands or reaction components, in a reaction medium at a first pH(A) at which the components are soluble;
(b) changing the pH(A) to a second pH(B) to cause a solid precipitate or a colloid of the copper oxo-hydroxide nanoparticle composition to be formed;
(c) separating, and optionally drying and/or formulating, the copper oxo-hydroxide nanoparticle composition produced in step (b).

29. A composition comprising ligand-modified copper oxo-hydroxide nanoparticles, wherein the copper oxo-hydroxide nanoparticles have a structure in which the one or more ligands are non-stoichiometrically substituted for the oxo or hydroxy groups, wherein the one or more ligands comprise a carboxylic acid ligand, or an ionised form thereof, as obtainable by the process according to claim 28.
